(19) United States
(12) Plant Patent Application Publication
(10) Pub. No.: US 2026/0283058 P1
(43) Pub. Date: Sep. 17, 2026

(54) Sweet Cherry Tree Named 'Media 51'

(50) ;

(71) Applicant: Cerasina GmbH, Kressbronn (DE)

(72) Inventor: Peter STOPPEL, Kressbronn (DE)

(73) Assignee: Cerasina GmbH, Kressbronn (DE)

(21) Appl. No.: 19/563,154

(22) Filed: Mar. 11, 2026

(30) Foreign Application Priority Data
Mar. 12, 2025 (DE) KIA 00130

(51) Int. Cl. A01H 6/74; A01H 5/08

(52) U.S. Cl. CPC A01H 6/7445; A01H 5/08
USPC PLT/181

(57) ABSTRACT

A new and distinct variety of sweet cherry tree, referred to by its cultivar name, ‘Media 51’, is described. The new plant variety produces large fruit with dark red skin, which are kidney-shaped, circular in ventral view, and have a low tendency for the skin to crack. The growth habit is wide spreading.

Latin name of genus and species of plant claimed: Prunus avium (L.) L.

Variety denomination: ‘Media 51’.

### CROSS REFERENCE TO RELATED APPLICATIONS

This application claims priority to Plant Breeders' Right Application No. KIA 00130, which was filed in Germany on Mar. 12, 2025, the contents of which are hereby incorporated by reference for all purposes.

### BACKGROUND OF THE INVENTION

The new plant variety of sweet cherry tree of the present invention was created by open pollination in Kressbronn, Baden-Wuerttemberg, Germany. The female parent TIETON™ ‘PC 7144-6’ (U.S. Plant Pat. No. 11,385). The male parent is unknown.

The seeds resulting from the above open pollination were stratified, germinated, and the resulting seedlings were planted in the field in Kressbronn, Germany. Small plants were obtained which were physically and biologically different from each other.

The new plant variety has been found to undergo asexual propagation by grafting onto GiSelA® 5 (‘GI 148/2’ U.S. Plant Pat. No. 9,622) rootstock in Kressbronn, Baden-Wuerttemberg, Germany. Asexual propagation techniques, such as grafting, have shown that the characteristics of the new plant variety are homogeneous, stable, and strictly transmissible by such asexual propagation from one generation to another. Accordingly, the new plant variety undergoes asexual propagation in a true-to-type manner.

### SUMMARY OF THE INVENTION

It was found that the new variety of sweet cherry tree of the present invention possesses a combination of characteristics of large fruit with dark red skin.

The new plant variety can be readily distinguished from its ancestors. In particular, fruit of the new plant variety is larger and ripen 10 days later than fruit of the seed parent ‘PC 7144-6’ and the new plant variety yield more fruit compared to ‘PC 7144-6’. Moreover, the new plant variety can be readily distinguished from non-parental related similar varieties. For example, the new plant variety produces kidney-shaped, larger fruit that ripens 8 days later compared to fruit of ‘Prim 25’ (U.S. Plant Pat. No. 33,719), which is heart-shaped. Also, the new plant variety exhibits a slightly more vigorous growth habit compared to ‘Prim 25’.

The new plant variety has been named the ‘Media 51’ variety. The new plant variety has also been referred to as Breeder's Reference: ‘Stop G112’

### BRIEF DESCRIPTION OF THE DRAWINGS

The accompanying photographs show as nearly true as it is reasonably possible to make the same, in a color illustration of this character, typical specimens of the new variety.

FIG. 1—illustrates ripe fruit growing on the new plant variety.

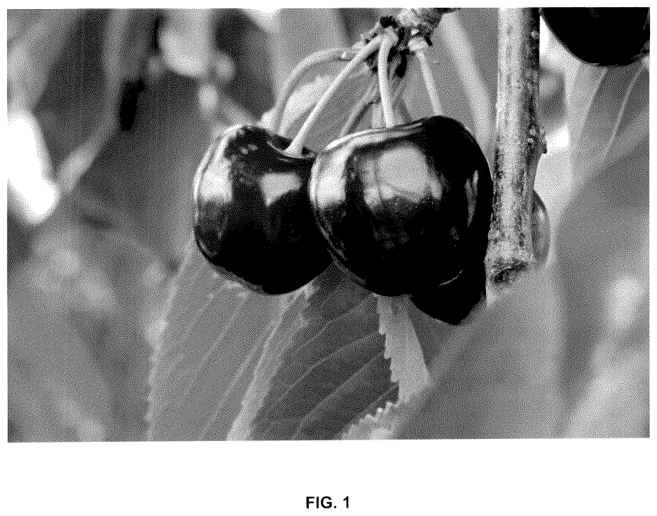

FIG. 2—illustrates a halved ripe fruit of the new plant variety.

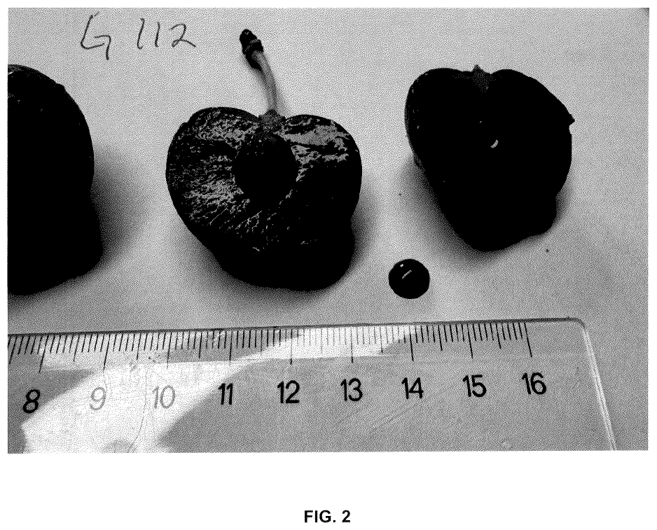

### DETAILED BOTANICAL DESCRIPTION

The chart used in the identification of colors is that of The Royal Horticultural Society (The R.H.S. Colour Chart, 2015 edition). The terminology which precedes reference to the chart has been added to indicate the corresponding color in more common terms. The R.H.S. Colour Chart designation used herein represents the closest color observed on the majority of the specified botanical features. The description is based on seven-years-old specimens of the new plant variety while growing on GiSelA® 5 ('GI 148/2′ U.S. Plant Pat. No. 9,622) rootstock in Kressbronn, Germany.
Botanical classification: Prunus avium (L.) L. cultivar ‘Media 51’.
Commercial classification: Sweet Cherry Tree.
Tree:
   Growth.—Approximately 50 cm per year.
   Height.—Approximately 3.0 m.
   Width.—Approximately 2.0 m.
   Growth habit.—Wide spreading.
   Trunk diameter.—Approximately 10 cm at 20 cm above ground.
   Trunk surface texture.—Smooth with prominent lenticels.
   Lenticel length.—Typically 1.0 cm to 2.0 cm.
   Lenticel width.—Approximately 0.5 cm.
   Lenticel color.—Greyed-Orange Group 167A.
   Lenticel density.—Medium.
   Bark color.—Grey-Brown Group 199A.
Branches:
   Diameter.—Typically 0.5 cm to 1.5 cm.
   Surface.—Smooth.
   Form.—Typical sweet cherry.
   Bark color.—Greyed-Orange Group 164A.
   Average angle.—Typically 60 degrees to 75 degrees.
   Lenticels length.—Approximately 1.0 mm.
   Lenticels width.—Approximately 1.0 mm.
   Lenticels shape.—Roundish.
   Lenticels color.—Greyed-Yellow Group 161B.
   Lenticels density.—Few, scattered.
Leaves:
   Length.—Typically 17.0 cm to 20.0 cm.
   Width.—Typically 8.0 cm to 10.0 cm.
   Form.—Long, narrow.
   Texture.—Leathery, no crisp feeling.
   Thickness.—Medium.
   Base.—Rounded.
   Apex.—Acute.
   Margin.—Serrations regular.
   Color mature leaves upper surface.—Green Group 143A.
   Color mature leaves lower surface.—Green Group 142D.
   Petiole shape.—Narrow.
   Petiole length.—Typically 3.5 cm to 4.5 cm.
   Petiole diameter.—Approximately 2.0 mm.
   Petiole color.—Adaxial Red-Purple Group 59A, abaxial Yellow-Green Group 149C.
   Venation type.—Pinnate.
   Veins color upper surface.—Yellow-Green Group 154D.
Flower buds:
   Length.—Approximately 10.0 mm.
   Width.—Approximately 4.0 mm.
   Color.—Reddish.
   Pedicel length.—Approximately 12.0 mm.
   Pedicel diameter.—Approximately 1.0 mm.
Flowers:
   Bloom timing.—Approximately Apr. 11, 2025.
   Blooming period.—The blooms are out for approximately 7 days.
   Number of flowers per raceme.—Typically 3 to 7.
   Fragrance.—None.
   Diameter.—Approximately 4.0 cm.
   Petals number.—5.
   Petals length.—Approximately 17.0 mm.
   Petals width.—Approximately 13.0 mm.
   Petals shape.—Oval, aspect slightly obcordate.
   Petals margin.—Slightly undulated.
   Petals texture and appearance.—Soft texture.
   Petals color when opening.—Upper and lower surfaces White N999D.
   Petals color fully opened.—Upper and lower surfaces White N999D.
   Sepals shape.—Conical.
   Sepals margin.—Regular.
   Sepals length.—Approximately 12.0 mm.
   Sepals width.—Approximately 5.0 mm.
   Sepals color.—Upper and lower surfaces are Yellow-Green Group 146C.
   Stamens number per flower.—Approximately 36.
   Filament length.—Typically 2.0 to 13.0 mm.
   Anthers shape.—Round-oval.
   Anthers length.—Approximately 1.0 mm.
   Anthers color.—Greyed-Orange Group N167A.
   Pollen color.—Greyed-Orange Group N167A.
   Pollen amount.—Plentiful.
   Pistils.—Approximately 19.0 mm.
   Style length.—Approximately 14.0 mm.
   Style color.—Yellow-Green Group 145B.
   Stigma shape.—Oval.
   Stigma color.—Yellow-Green Group 145B.
Fruit:
   Shape.—Kidney shaped; circular in ventral view.
   Diameter.—Typically between 30.0 mm to 32.0 mm.
   Weight.—Approximately 18 g.
   Pistil end.—Stump.
   Fruit stalk length.—Approximately 25.0 mm.
   Fruit stalk thickness.—Approximately 2.0 mm.
   Skin color.—Greyed-Purple Group 187A.
   Skin ground color.—Red Group 53A.
   Skin thickness.—Medium.
   Skin texture.—Smooth.
   Skin tendency to crack.—Low.
   Flesh color.—Greyed-Purple Group 184D.
   Firmness.—Firm.
   Aroma.—Pleasant.
   Eating quality.—Good.
   Stone length.—Approximately 12.0 mm.
   Stone width.—Approximately 9.0 mm.
   Stone form.—Oval.
   Stone color.—Greyed-Orange Group 173D.
   Stem length.—Typically 35.0 mm to 40 mm.
   Stem width.—Approximately 1.0 mm.
   Stem color.—Yellow-Green Group 146C.
Development:
   Productivity.—Approximately 18 kg of fruit per tree.
   Shipping/storability of fruit.—Good quality for commercial cultivation; good transportability and storability of the fruit.
   Resistance to pathogens.—Cherry leaf roll virus (Nepovirus), Prune dwarf virus (Ilarvirus), Prunus necrotic ringspot virus (Ilarvirus), Tomato black ring virus (Nepovirus (subgroup B)), Tomato ringspot virus (Nepovirus lycopersici), Little cherry virus 1 (Velarivirus nanoavii), Little cherry virus 2 (Ampelovirus nanoavii).

The new ‘Media 51’ variety has not been observed under all possible environmental conditions to date. Accordingly, it is possible that the phenotypic expression may vary somewhat with changes in light intensity and duration, cultural practices, and other environmental conditions.

I claim:

1. A new and distinct variety of sweet cherry tree named ‘Media 51’ substantially as illustrated and described herein.
